(19) United States
(12) Patent Application Publication
(10) Pub. No.: US 2017/0234853 A1
(43) Pub. Date: Aug. 17, 2017

(54) METHOD FOR DETERMINING THE STRUCTURAL PROFILE OF A FIBRIN CLOT REFLECTING THE STABILITY THEREOF, IN ORDER TO PREDICT THE RISK OF BLEEDING, THROMBOSIS OR RETHROMBOSIS

(71) Applicants: DIAGNOSTICA STAGO, Asnieres S/seine (FR); CENTRE NATIONAL DE LA RECHERCHE SCIENTIFIQUE- CNRS, Paris (FR); UNIVERSITE GRENOBLE I JOSEPH FOURIER, Saint Martin D'heres (FR); CENTRE HOSPITALIER UNIVERSITAIRE DE GRENOBLE, La Tronche (FR)

(72) Inventors: Geneviève CONTANT, Courbevoie (FR); Benoît POLACK, Saint Martin Le Vinoux (FR); François CATON, Saint Martin D'Heres (FR); Carhel DASSI, Grenoble (FR)

(21) Appl. No.: 15/326,120

(22) PCT Filed: Jul. 24, 2015
(86) PCT No.: PCT/FR2015/052050

§ 371 Date: Jan. 13, 2017

(30) Foreign Application Priority Data
Jul. 25, 2014 (FR) 14 57219

(51) Int. Cl. G01N 33/49; G01N 21/31

(52) U.S. Cl. CPC G01N 33/4905; G01N 21/31; G01N 2201/12; G01N 2333/7454; G01N 2333/75; G01N 2800/224; G01N 2800/50

(57) ABSTRACT

The present invention relates to a method for dynamically determining the structural profile of a fibrin clot, reflecting the stability thereof in a biological sample of a patient. The method preferably includes a step that makes it possible to predict the risk of bleeding, thrombosis or rethrombosis and to select the anticoagulant that is best suited to the clinical situation of a patient.

The present invention relates to a method for dynamically measuring the structure of a fibrin clot reflecting its stability in a biological sample.

Blood coagulation is a complex phenomenon which involves several factors, including in particular:
tissue factor, which is responsible for the generation of thrombin, and
fibrinogen, which is converted to fibrin under the activation of thrombin.

Fibrin, through its accumulation, results in the formation of a clot, which stops the hemorrhage.

Blood coagulation is regulated by various compounds, including plasminogen, responsible for plasmin generation. Indeed, the clot formed by fibrin is lysed under the effect of plasmin: this is fibrinolysis. Fibrinolysis plays an important role in clot stability and hemorrhagic or thrombotic risk, if it begins after clot formation. If it is initiated at the same time, there is competition between thrombin-fibrin binding and plasmin-fibrin binding, and thus between fibrin formation and lysis, depending on the endogenous fibrinolytic capacity in the tissue concerned.

A very large number of methods for evaluating the hemorrhagic or thrombotic risk of a patient have been described for a long time. These methods are based on thrombin generation (TG) and/or plasmin generation (PG), on fibrinolysis, on the viscoelastic properties of the clot (thromboelastography or TEG) or on its optical properties (turbidimetry). Among the available methods, some, such as the partial thromboplastin (PTT or aPTT), comprise measuring the viscosity in an electromagnetic method, and are not accurate for determining the clinical seriousness associated with hemorrhagic or thrombotic risk (J J. van Veen, Br J Haematol 2008; M. Adams, Semin Thromb Hemost 2009; M. Smid, J Thromb Haemost 2011; E. Castoldi, Thromb Res 2011; O. R. Zekavat, Clin and Applied Thromb Haemost 2013). The thrombin generation assay (TGA) is recognized as a versatile diagnostic tool for investigating patients with hypocoagulable and hypercoagulable phenotypes. With regard to the evaluation of the thrombin potential by means of an optical method, it requires removing the fibrinogen and the platelets from the sample. The measurement of TG by a chromogenic method or by fluorescence requires the use of a dedicated instrument due to the long measuring time (90 min), and specific software for analyzing the signal and interpreting it;

consequently, it is used in research.

TG stops at thrombin; it reflects neither the stability of the fibrin clot nor its resistance to lysis. Measurement of the quality of the fibrin clot coupled to TG provides additional information for evaluating the efficacy of a treatment (Y Dargaud, Haemophilia 2011). In particular, the structure of the clot is an important determinant in hemorrhagic and thrombotic risk (Wolberg, Blood Reviews 2007; Wolberg et al., Transfus Apher Sci 2008). Determination of the viscoelastic properties of the clot during coagulation by thromboelastography using electronic measurement (TEG) or optical measurement (ROTEM) is used in real time for perioperative transfusion or clinical decisions. However, this method suffers from its well-known lack of reproducibility and is not suitable for the routine management of several samples.

With regard to the other methods, they use a specific and bulky equipment, and their implementation is limited to research laboratories, since the tests cannot be automated and require a large volume of samples.

In particular, a test for simultaneously measuring thrombin generation and plasmin generation using two fluorescent substrates (Novel Haemostasis Assay NHA) has been described, but can only be carried out on a TG-dedicated fluorimeter (A. van Geffen, Hematology 2011, WO2011/057143). The combination of the two tests, TGA and TEG, modified by the addition of t-PA to reflect the quality of the fibrin clot provides additional information (Y Dargaud, Haemophilia 2011), but its implementation can only be performed on 2 specific instruments and is not practical. The available structural methods (electron microscopy, confocal microscopy, electrophoretic light scattering, small-angle X-ray scattering (SAXS) or small-angle neutron scattering (SANS)) can be used to monitor the effect of the structure of the fibrin clot on the sensitivity to fibrinolysis, but these methods cannot be automated and are lengthy and complex to carry out.

The available turbidimetric and light scattering methods are limited to the fibrinogen and thrombin purified system.

Recently, a physical method of multi-wavelength spectrophotometry has been proposed for quantitatively determining the nanostructure of the fibrin clot (including the radius of the fibers, the number of protofibrils per fiber, the inter-protofibril distance and the density of the fibers) during clot formation, but this method is manual, requires a large sample volume (600 μl), and does not lend itself to automation by virtue of the long measuring time (90 min) and the lack of availability of all the wavelengths on the routine instrument (C. Yeromonahos, Biophysical J 2010; Thesis Oct. 12, 2011, and ATVB 2012).

A large number of studies have shown that fundamental differences exist in the formation of the fibrin clot between tests in a purified system (thrombin+fibrinogen) and situations where the thrombin is generated in situ, since the presence of cells and of other proteins modifies the TG and the fibrin formation.

Finally, the analysis of the optical profile of fibrin formation (or “waveform analysis”) can be carried out, but it remains qualitative and laborious.

No prior art method is thus capable of measuring fibrin clot stability, in order to predict the risk of bleeding, thrombosis or rethrombosis, in common practice. The majority of these methods are reserved for use in research or at the patient's bedside because:
they are limited to a purified sample because of the interference of the plasma fibrinogen, or to the fibrin formation step after addition of thrombin to the plasma,
they require specialized equipment, a large amount of plasma, and a great deal of expertise,
they lack reproducibility and practicality and are too lengthy to carry out, and/or
they do not reflect the stability of the fibrin clot or its resistance to lysis.

Thus, there is a need for a method for determining the structural profile of a fibrin clot reflecting its stability using a sample from a patient, said method being capable of predicting the hemostatic risk of said patient, since only the method of investigation by confocal microscopy, considered as the standard method at the present time, allows this (J P. Collet, Arterioscler Thromb Vasc Biol. 2000).

Such a method should also make it possible to quantitatively measure the fibrinolysis in the sample, and must be reliable, automated, reproducible, simple and fast to carry out.

The inventors have developed a method capable of predicting the hemostatic risk and of quantitatively measuring the fibrinolysis in a nonpurified sample. This method also allows the automated determination of the structure of the fibrin clot. It is reliable, simple and fast to carry out, and reproducible. Finally, it adds to the information given by thrombin generation with the stability of the fibrin clot.

The present invention thus relates to a method for determining the structural profile of a fibrin clot, reflecting the stability thereof in a biological sample from a patient, said method comprising the following steps:
a) mixing the undiluted biological sample with some tissue factor and phospholipids. The tissue factor and the phospholipids can optionally be in a mixture with a plasminogen activator, preferably the tissue plasminogen activator (t-PA);
b) incubating, in particular at 37° C., the mixture obtained in step a), then adding calcium ions to the mixture obtained, in order to initiate the formation of a clot;
c) measuring the turbidity or the optical density of the clot in the process of being formed in step b), at at least two wavelengths of between 450 nm and 850 nm, and for a time of between 1 and 35 minutes; and
d) determining the profile of the clot analyzed in c), preferably expressed as number of protofibrils, density and radius, by means of the formula τ.λ⁵=A [Fg].(λ²−B), where τ is the turbidity of the clot or the expression of the optical density in turbidity, at a given wavelength λ, [Fg] is the initial weight concentration of fibrinogen, and A and B are coefficients proportional to the density and the radius of the fibers constituting the clot, respectively.

Such a method makes it possible to predict the risk of bleeding, thrombosis or rethrombosis, for the patient concerned, this being in a very short time (i.e. less than one hour) as described in particular in Example 6. Indeed, the number of protofibrils of the various normal, hypo- and hypercoagulant, and hypo- and hyperfibrinolytic plasmas makes it possible to distinguish these plasmas in 30 minutes on the basis of their structural profile during thrombin generation, fibrin formation and lysis.

The differentiation between the hypercoagulant, normal and hypocoagulant profiles is based on the number of protofibrils, the time to reach the plateau and the rate at which the plateau is reached.

The protofibril plateau, which reflects the stability of the clot, lasts for more than 20 minutes, for 10 to 20 minutes and for less than 10 minutes, respectively, for a hypercoagulant, normal and hypocoagulant profile, in the presence of tissue factor and t-PA. The protofibril plateau is shortened proportionally to the concentration of anti-FXa anticoagulant (rivaroxaban) in the hypocoagulant plasma, and is extended proportionally to the concentration of inhibitor of plasminogen activator (PAI-1) in the hypofibrinolytic plasma.

The downward slope of the protofibril plateau is greater the faster the lysis; this is the case with hyperfibrinolytic plasmas (deficient in PAI-1 or containing rivaroxaban). On the other hand, it is zero when the clot is resistant to lysis; this is the case with hypercoagulant plasmas (deficient in protein S or in tissue factor inhibitor TFPI).

As shown in the Examples, the disappearance of the protofibrils, corresponding to total lysis, is reached in 33 minutes for all the samples, except for one of the hypercoagulant plasmas, deficient in protein S.

The term “profile of a fibrin clot” is intended to mean the structure of said clot reflecting the stability thereof. The method according to the invention makes it possible to determine the structure of the clot formed, and in particular the number of protofibrils constituting the clot, their density and their radius.

The method according to the invention uses a simple biological sample from the patient. This biological sample is used undiluted in the method. Preferably, the biological sample is a blood sample, a plasma sample, a sample of platelet-rich plasma or of platelet-poor plasma or a sample of plasma containing platelet microparticles, erythrocytes or any other cell. Preferably, the biological sample is a sample of platelet- poor plasma (PPP). In this case, it is, in particular, obtained by centrifugation of the citrated tube comprising the patient's blood sample for 15 minutes, at a speed of from 2000 to 2500 g, in a thermostatic centrifuge at 18-22° C. If the sample must be stored, the following protocol can be used:
the plasma is rapidly decanted, leaving approximately 0.5 cm of plasma above the cell layer of white blood cells and platelets;
the plasma is recovered in a hemolysis tube or a plastic tube;
this tube is again centrifuged for 15 minutes, at a speed of from 2000 to 2500 g, in a thermostatic centrifuge at 18-22° C.;
fractions of from 0.5 to 1 ml are aliquotted without taking the bottom of the tube (cell debris); then
they are rapidly frozen at −70° C., better still at −80° C.

The undiluted biological sample is used in the present invention in a small volume. Preferably, the biological sample has a volume of between 5 μl and 500 μl, preferably of between 50 μl and 400 μl, preferably of between 50 μl and 300 μl, preferably of between 100 μl and 300 μl, preferably of approximately 200 μl. Such a volume is in fact sufficient for routine instrument analysis, but may be reduced provided that the ratio of sample volume to final volume is adhered to (i.e. ratio of approximately 2:3). It can be further reduced in the case of a microsystem for use at the patient's bedside, provided that this volume ratio constraint is adhered to.

Step a) comprises mixing the undiluted biological sample with tissue factor and phospholipids. The undiluted biological sample is preferably used as is, in the case of plasma. It is mixed with tissue factor (TF), preferably human tissue factor, and phospholipids (PLs), which are preferably semi-purified or purified.

Said tissue factor and the PLs are preferably premixed with a solution of plasminogen activator, in particular with an affinity for fibrin, preferably tissue plasminogen activator (t-PA), then the whole mixture is added to the undiluted biological sample. In this case, the TF and the phospholipids are first reconstituted in solution by mixing with a solution of t-PA, then the mixture obtained is added to the undiluted biological sample.

The plasminogen activator preferably used has an affinity for fibrin. Preferably, the plasminogen activator used is t-PA, in particular alteplase, sold under the reference Actilyse® by Boehringer Ingelheim. Any activator with an affinity for fibrin, such as fibrin-specific streptokinase derivatives, may be used.
The mixture of step a) may comprise a phospholipid concentration of from 2 to 5 μM in the mixture, preferably a concentration of approximately 4 μM.

Preferably, the TF is used in an amount such that its final concentration in the mixture with the undiluted biological sample is between 0.01 and 20 μM.

Preferably, the TF is used in an amount such that its final concentration in the mixture with the undiluted biological sample is between 0.1 and 5 pM, preferably between 1 and 5 pM.

When t-PA is present, it is used in an amount such that its final concentration in the mixture with the TF and the undiluted biological sample is between 0.1 and 0.3 μg/ml, preferably between 0.1 and 0.2 μg/ml.

Preferably, the mixing of the tissue plasminogen activator (t-PA) in the tissue factor (TF) of step a) is carried out in a [t-PA/TF] respective weight ratio of between 800 and 1700, preferably of between 1000 and 1300. The tissue factor and the t-PA were used at the final concentrations of 2 pM and 150 ng/ml respectively in Example 6, for distinguishing the various samples. Preferably, the t-PA and the TF are used in a ratio of from 75 to 150 ng/ml oft-PA for 0.1 to 5 pM of TF, preferably for 1 to 5 pM of TF.

The mixture of step a) may also comprise at least one divalent cation, preferably calcium ions. The calcium ions may be present at a final concentration of from 15 to 20 mM, preferably a final concentration of 17 mM.

Preferably, step a) of the method according to the invention comprises:
a1) introducing TF into a solution of phospholipids and optionally calcium, then
a2) mixing the solution obtained in al) with the undiluted biological sample.

Alternatively, preferably, step a) of the method according to the invention comprises:
a1) introducing t-PA into a solution of TF,
a2) adding phospholipids and optionally adding calcium to the solution obtained in al), then
a3) mixing the solution obtained in al) or a2) with the undiluted biological sample.

At the end of step a), a mixture of at least the TF with the undiluted biological sample is obtained for determining the structural profile during coagulation and a mixture of at least the TF and t-PA with the undiluted biological sample is obtained for determining the stability profile of the clot and the structural profile during lysis.

There then follows a step b) of incubating the mixture obtained in step a), then adding at least one divalent cation, preferably calcium ions, to the mixture obtained, in order to initiate thrombin generation and the formation of a clot. Preferentially, step b) initiates thrombin generation and the formation of a clot.

Step b) thus comprises incubating the mixture obtained in step a). This incubation can typically be carried out for a time of between 60 seconds and 400 seconds, preferably of between 200 seconds and 350 seconds, preferably of 300 seconds on the instrument, at a temperature of between 30° C. and 40° C., preferably of approximately 37° C. This incubation time can be shortened with a reduction of the volumes, in particular with the use of a microsystem. Divalent cations, preferably calcium ions, are then added to the mixture incubated. These calcium ions may be added in the form of a CaCl₂ solution, at a concentration of approximately 0.1 M.

The thrombin generation and the formation of a fibrin clot are initiated through the addition of a divalent cation such as calcium, in the presence of TF.

The structure of the fibrin clot is then monitored dynamically, that is to say every 2 seconds or less during step c), by measuring the turbidity at at least two wavelengths of between 450 nm and 850 nm, and for a time of between 1 and 35 minutes. Preferably, the turbidity measurement is carried out at least at the two wavelengths closest to the extreme values, for example 540 nm and 780 nm, or better still 540 nm and 760 nm. Preferably, the turbidity measurement is carried out simultaneously at at least two wavelengths which are the most optimal for the measurements carried out, for example simultaneously at 540 nm and 780 nm, or better still simultaneously at 540 nm and 760 nm. Preferably, the measuring time of step c) is between 1 minute and 35 minutes, preferably between 10 and 35 minutes, for both the coagulation profile and the lysis profile; it is preferably approximately 15 minutes for the coagulation profile (TF alone) and approximately 30 minutes for the lysis profile (TF+t-PA).

During this time, the structure of the fibrin clot is thus analyzed, in particular through the variable protofibril formation, radius and density.

The structure of the fibrin clot may also be monitored dynamically during step c), by measuring the optical density at at least two wavelengths of between 450 nm and 850 nm, and for a time of between 1 and 35 minutes. Preferably, the optical density measurement is carried out at at least two wavelengths closest to the extreme values, for example 540 nm and 780 nm, or better still 540 nm and 760 nm. Preferably, the optical density measurement is carried out simultaneously at at least two wavelengths that are the most optimal for the measurements carried out, for example simultaneously at 540 nm and 780 nm, or better still simultaneously at 540 nm and 760 nm. Preferably, the measuring time of step c) is between 1 minute and 35 minutes, preferably between 10 and 35 minutes, for both the coagulation profile and the lysis profile; it is preferably approximately 15 minutes for the coagulation profile (TF alone) and approximately 30 minutes for the lysis profile (TF +t-PA).

The expression of τ at time t, at a given wavelength λ, applicable with an optical density measurement, is logarithmic according to the formula τ(t)=[OD(t)−OD(ini)]ln (10), where OD(t) and OD(ini) are respectively the optical density at time t and the initial optical density (at t=0). The determination of the profile of the clot analyzed in c) is obtained using a model linking the structure of the clot to its optical properties, preferably by the formula τ.λ⁵=A[Fg].(λ²−B), where τ is the turbidity of the clot at a given wavelength λ, [Fg] is the initial weight concentration of fibrinogen, and A and B are coefficients proportional to the density and to the radius of the fibers constituting the clot, respectively.

This turbidity or optical density measurement can be carried out using any existing instrument, and in particular using a turbidimeter or a spectrophotometer. Preferably, this measurement is carried out kinetically on an automated diagnostic device, preferably a coagulation analyzer. More preferentially, this measurement is carried out on the STA-R® Evolution Expert Series automated device from the Stago group. It can also be used with an optical microsystem.

In fine, the profile of the clot studied in step c) is determined by means of a model linking the structure of the clot to its optical properties. Preferably, this model links the turbidity to the structure by means of the formula (C. Yeromonahos, Biophysical J 2010):

τ.λ⁵=A[Fg].(λ²−B)

where:
τ is the turbidity of the clot at a given wavelength λ,
[Fg] is the initial weight concentration of fibrinogen, and
A and B are coefficients proportional to the density and to the radius of the fibers constituting the clot, respectively.

Thus, during step d), information is obtained regarding the structure of the fibrin clot, and thus regarding its properties, as a function of time.

With this information, monitoring of the structure of the clot during coagulation and fibrinolysis, reflecting the stability thereof, is obtained. It is thus possible to predict the risk of bleeding, thrombosis or rethrombosis of the patient whose biological sample was analyzed, all the more so since the intrinsic fibrinogen concentration of the patient, which is a known hemostatic risk factor, is taken into account.

The term “rethrombosis” is used in the case where the patient has already suffered a thrombosis, and is the subject of a specific medical follow-up to avoid recurrences.

In particular, at least steps c) and d) of the method according to the invention are carried out on an automated diagnostic device, preferably a coagulation analyzer.

More preferentially, all the steps of the method according to the invention are carried out on such an automated device. More preferentially, the method according to the invention is carried out on the STA-R® Evolution Expert Series automated device from the Stago group. Such an automated device makes it possible to simultaneously load the samples, to prepare the mixtures and carry out incubation, to measure the optical densities at at least two wavelengths and to determine the profile of the clot obtained from several samples simultaneously. The whole process is carried out in approximately 15 minutes for the coagulation and approximately 30 minutes for the coagulation followed by fibrinolysis. This makes it a fast, reliable, reproducible method which predicts the hemorrhagic or thrombotic risk in a patient. The method can be used on a routine instrument and supplements the information given by the thrombin generation with the structure of the fibrin clot, which is a reflection of the stability thereof, whereas this structure is available only by confocal microscopy and is qualitative.

A subject of the present invention is also a method for predicting the risk of bleeding, thrombosis or rethrombosis using a biological sample from a patient, said method comprising the following steps:
a) mixing the undiluted biological sample with tissue factor and phospholipids;
b) incubating the mixture obtained in step a), then adding calcium ions to the mixture obtained, in order to initiate the formation of a clot;
c) measuring the turbidity or the optical density of the clot being formed in step b), at at least two wavelengths of between 450 nm and 850 nm, and for a time of between 1 and 35 minutes; and
d) determining the profile of the clot analyzed in c) by means of the formula

τ.λ⁵=A[Fg].(λ²−B),

where τ is the turbidity of the clot or the optical density calculated according to the formula τ(t)=[OD(t)−OD(ini)]ln (10) at a given wavelength λ, [Fg] is the initial weight concentration of fibrinogen, and A and B are coefficients proportional to the optical density and to the radius of the fibers constituting the clot, respectively, and
e) comparing the profile obtained in d) with a control.

Steps a) to d) are as described above. In particular, step a) may also comprise adding plasminogen activator, preferably tissue plasminogen activator, as is described above.

Step e) corresponds to a comparison between the profile obtained for the biological sample from the patient under consideration, and a profile obtained for a control. Said control may be, in particular:
a biological sample from one or more healthy individuals, preferably a reference plasma,
a biological sample from one or more quality controls so as to mimic the conditions of patients. This may be one or more quality controls produced from a pool of normal plasmas which are untreated or have been treated to make the plasma deficient in a given factor of coagulation or fibrinolysis so as to mimic the conditions of patients, as in Example 1,
a biological sample from a hemorrhagic patient,
a biological sample from a thrombotic patient, or
a biological sample from a patient having suffered rethrombosis, in particular as in Example 6.

This simple step e) makes it possible to directly and rapidly deduce the risk of thrombosis or hemorrhage of the patient under consideration, all the more so since the intrinsic fibrinogen concentration of the patient, which influences the stability of the clot, is taken into account, as shown in Example 6.

Preferably, such a method comprises, following step e), a step f) of selecting the anticoagulant best suitable for the clinical situation of said patient, said clinical situation being chosen from atrial fibrillation or any other cardiac impairment; a cancer or any other malignant condition or precancerous state; and a risk of venous or arterial thrombosis. Preferably, the clinical situation of said patient is chosen from atrial fibrillation; a cancer; and a risk of venous or arterial thrombosis.

Among the anticoagulants that can be used, heparin (unfractionated heparin or low-molecular-weight heparin) and derivatives thereof (such as fondaparinux, idraparinux, enoxaparin, tinzaparin, nadroparin); vitamin K antagonists (VKAs) such as coumarin derivatives (such as acenocoumarol, warfarin or fluindione); thrombin inhibitors such as hirudin, bivalirudin, ximelagatran, dabigatran or odiparcil; factor Xa inhibitors such as rivaroxaban, otamixaban, apixaban, betrixaban or edoxaban, are preferably chosen.

VKAs are used in the prevention of strokes and systemic embolisms in patients suffering from nonvalvular atrial fibrillation. Direct oral anticoagulants (thrombin inhibitors or factor Xa inhibitors) make it possible to avoid the initial adaptation phase of VKAs, the monitoring of which is restrictive and has a high hemorrhagic risk. They also make it possible to avoid prescribing parenteral injections of low-molecular-weight heparins, making the ambulatory treatment of deep thrombosis and pulmonary embolism easier. They are an alternative to heparin treatment for young subjects, with neither renal nor hepatic failure, in the prevention of venous thromboembolic disease in orthopedic surgery. The method makes it possible to select the anticoagulant most suitable for the clinical situation of said patient since the main adverse effects of the new molecules relate to hemorrhagic events (especially the digestive sphere and rather in medical indications) and then thromboembolic events, rather in the aftermath of surgery.

The treatment of thromboembolic events in patients suffering from cancer is based on low-molecular-weight heparins, administered, without sequential oral administration, for three to six months; they notably reduce thromboembolic recurrences by 50%, without increasing hemorrhages. The method makes it possible to select the low-molecular-weight heparin and the dose most suitable for said patient since:
i) the incidence of thromboembolic event recurrences under treatment remains high; it is about 7% after three or six months of treatment. It is higher in the presence than in the absence of cancer,
ii) in the event of recurrence, the dose of low-molecular-weight heparin can be increased by 10%, since it could be effective and well-tolerated.

The following examples are given with a view to illustrating various embodiments of the invention.

The figure legends are given below.

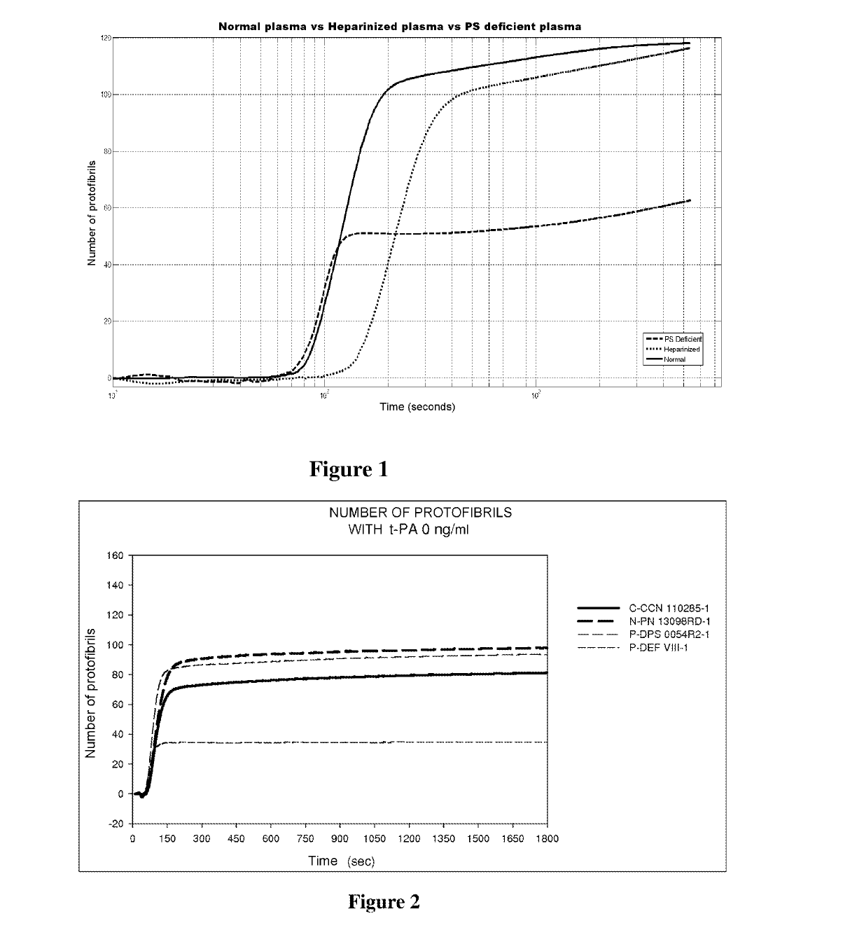

FIG. 1: Measurement of the number of protofibrils as a function of time for a normal plasma, for a hypocoagulant plasma (heparinized control plasma 0.2 IU/ml) and for a hypercoagulant plasma (control plasma deficient in protein S).

FIG. 2: Measurement of the number of protofibrils as a function of time for normal plasmas (control plasma CCN and normal pool PN), hypocoagulant plasma (plasma congenitally deficient in FVIII:C DEF VIII) and hypercoagulant plasma (plasma deficient in protein S DPS).

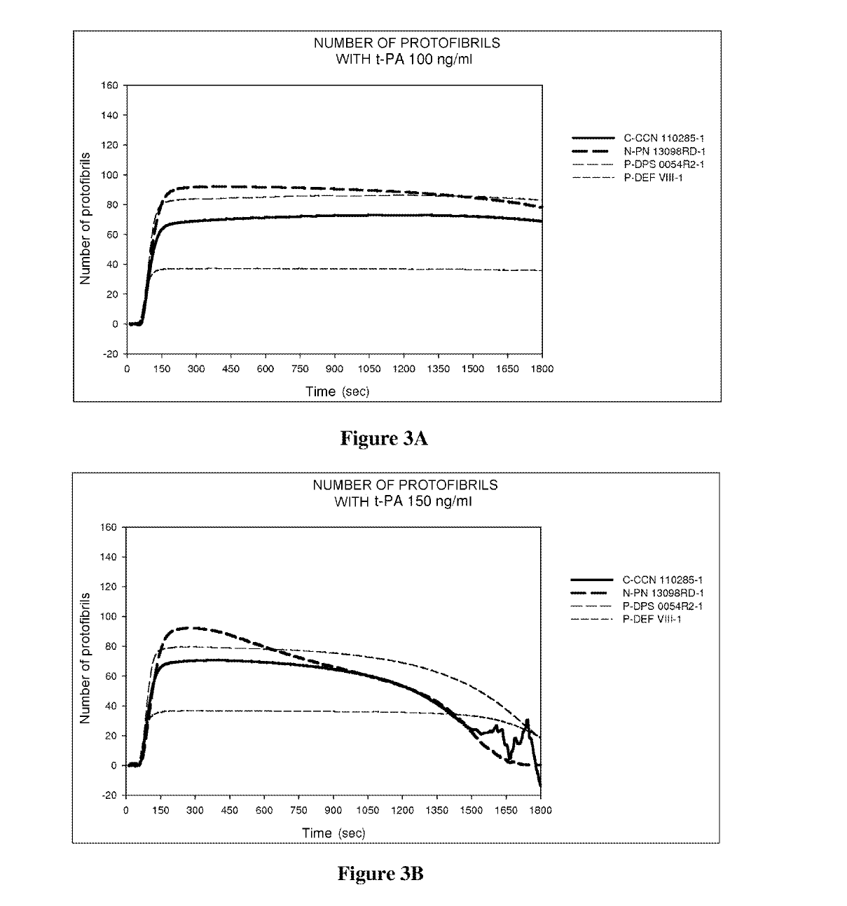

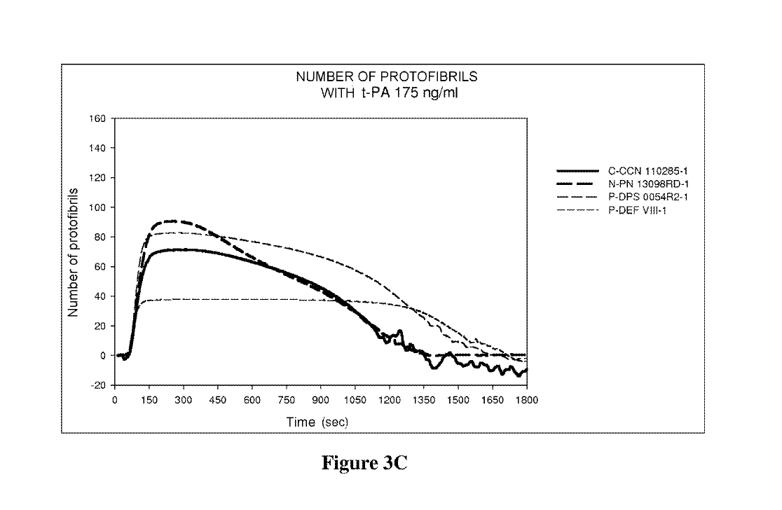

FIG. 3: Measurement of the number of protofibrils as a function of time for normal plasmas (control plasma CCN and normal pool PN), hypocoagulant plasma (plasma congenitally deficient in FVIII:C DEF VIII) and hypercoagulant plasma (plasma deficient in protein S DPS).
A: t-PA at 100 ng/ml;
B: t-PA at 150 ng/ml;
C: t-PA at 175 ng/ml.

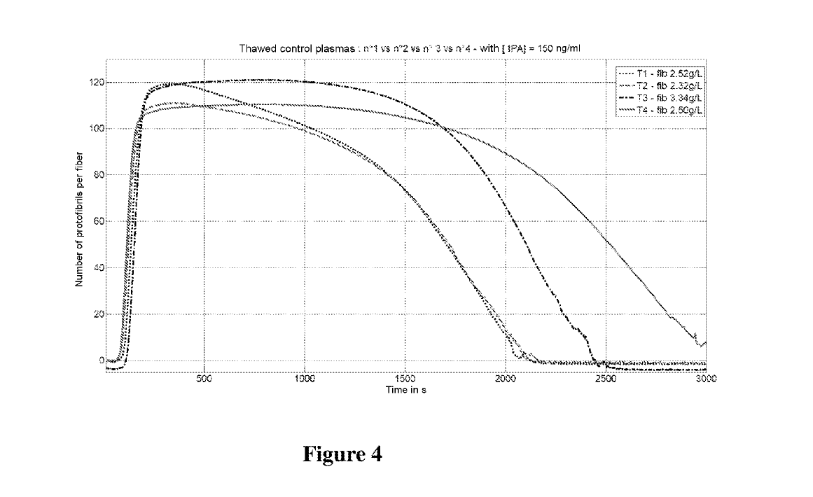

FIG. 4: Measurement of the number of protofibrils as a function of time for 4 normal control plasmas comprising 2.52 g/l, 2.32 g/l, 3.34 g/l and 2.50 g/l of fibrinogen, respectively, with t-PA at 150 ng/ml.

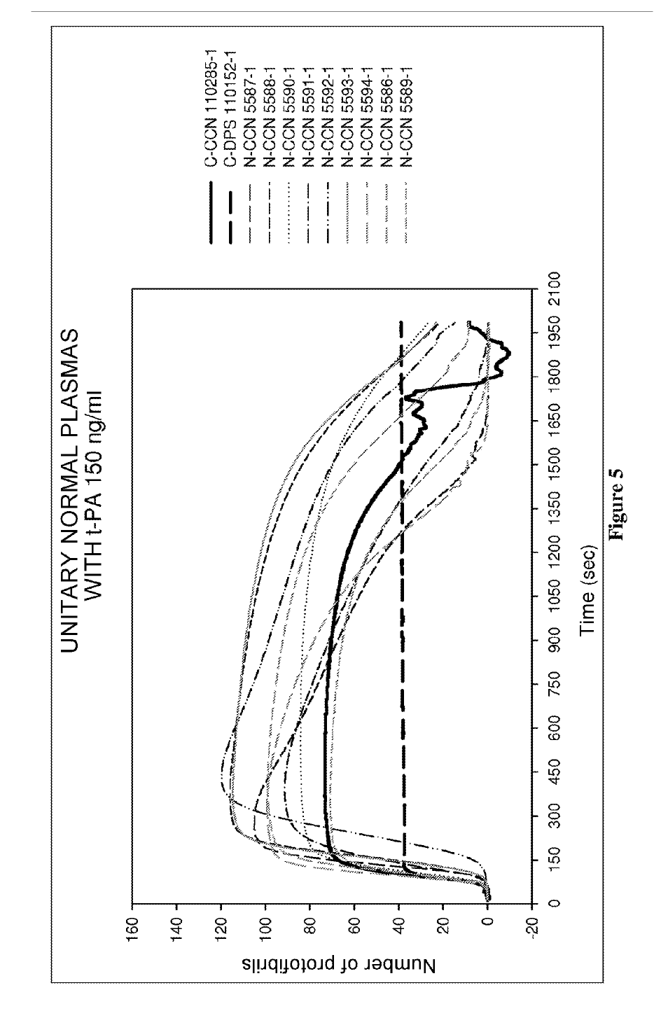

FIG. 5: Measurement of the number of protofibrils as a function of time for 9 control plasmas (plasmas from N-CCN 5587 to N-CCN 5594), a normal control plasma (C-CCN) and a hypercoagulant control plasma (C-DPS) with t-PA at 150 ng/ml.

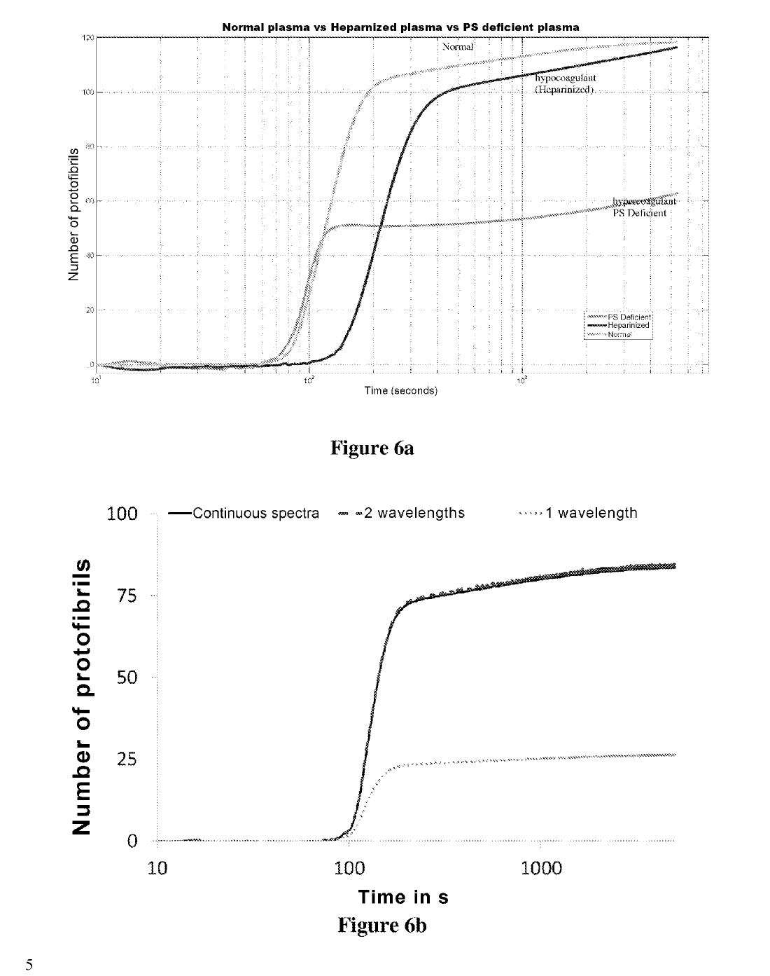

FIG. 6:
6a) Measurement of the number of protofibrils for a normal plasma (Normal), a hypocoagulant plasma (heparinized control plasma 0.2 IU/ml) and a hypercoagulant plasma (plasma deficient in protein S) at 2, 3, 7 or 20 wavelengths as a function of time,
6b) Measurement of the number of protofibrils for a normal control plasma as a function of time, at the wavelength of 540 nm, and at the two optical wavelengths of 540 nm and 780 nm and in continuous spectrum.

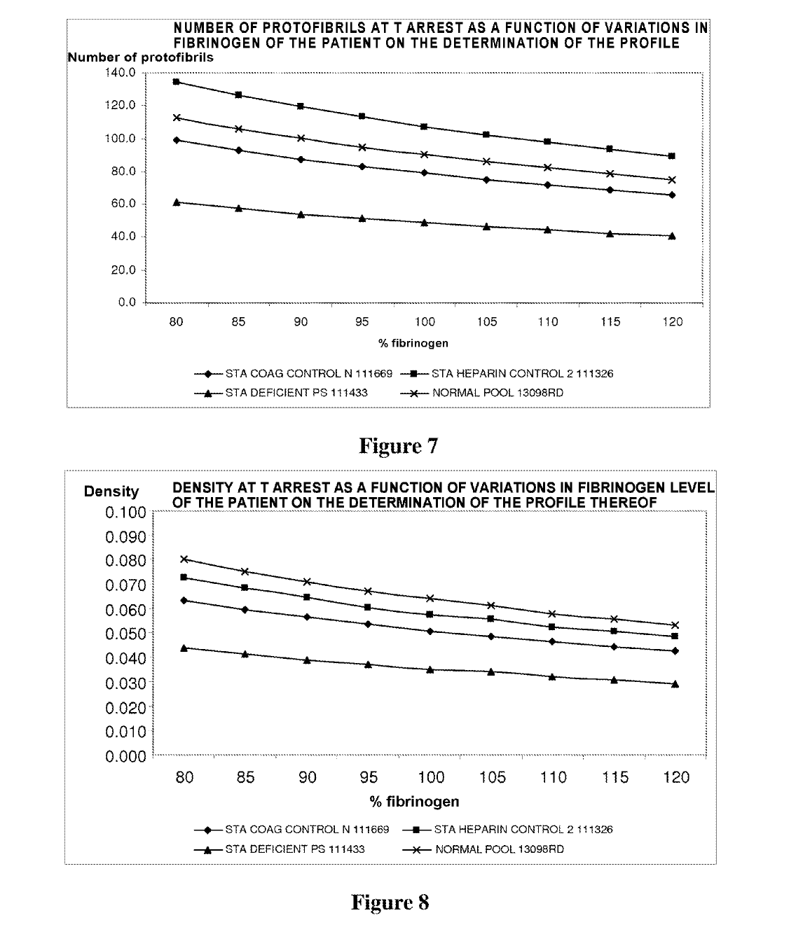

FIG. 7: Measurement of the number of protofibrils at the time of arrest, for a normal control plasma (STA COAG CONTROL N), a normal pool (NORMAL POOL), a hypocoagulant control plasma (STA HEPARIN CONTROL 2) and a hypercoagulant control plasma (STA DEFICIENT PS), as a function of the variations in percentage fibrinogen of the patient over the determination of the profile thereof

FIG. 8: Measurement of the density of protofibrils at the time of arrest, for a normal control plasma (STA COAG CONTROL N), a normal pool (NORMAL POOL), a hypocoagulant control plasma (STA HEPARIN CONTROL 2) and a hypercoagulant control plasma (STA DEFICIENT PS), as a function of the variations in percentage of fibrinogen of the patient over the determination of the profile thereof.

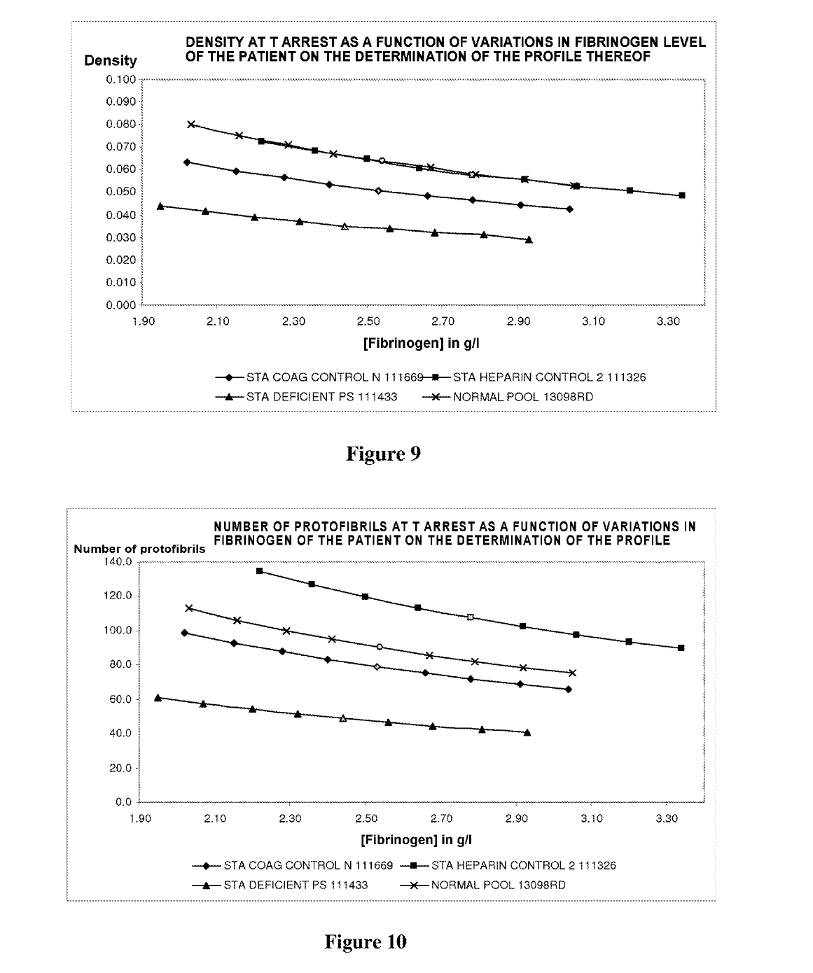

FIG. 9: Measurement of the density of protofibrils at the time of arrest, for a normal control plasma (STA COAG CONTROL N), a normal pool (NORMAL POOL), a hypocoagulant control plasma (STA HEPARIN CONTROL 2) and a hypercoagulant control plasma (STA DEFICIENT PS), as a function of the variations in level of fibrinogen of the patient over the determination of the profile thereof.

FIG. 10: Measurement of the density of protofibrils at the time of arrest, for a normal control plasma (STA COAG CONTROL N), a normal pool (NORMAL POOL), a hypocoagulant control plasma (STA HEPARIN CONTROL 2) and a hypercoagulant control plasma (STA DEFICIENT PS), as a function of the variations in level of fibrinogen of the patient over the determination of the profile thereof.

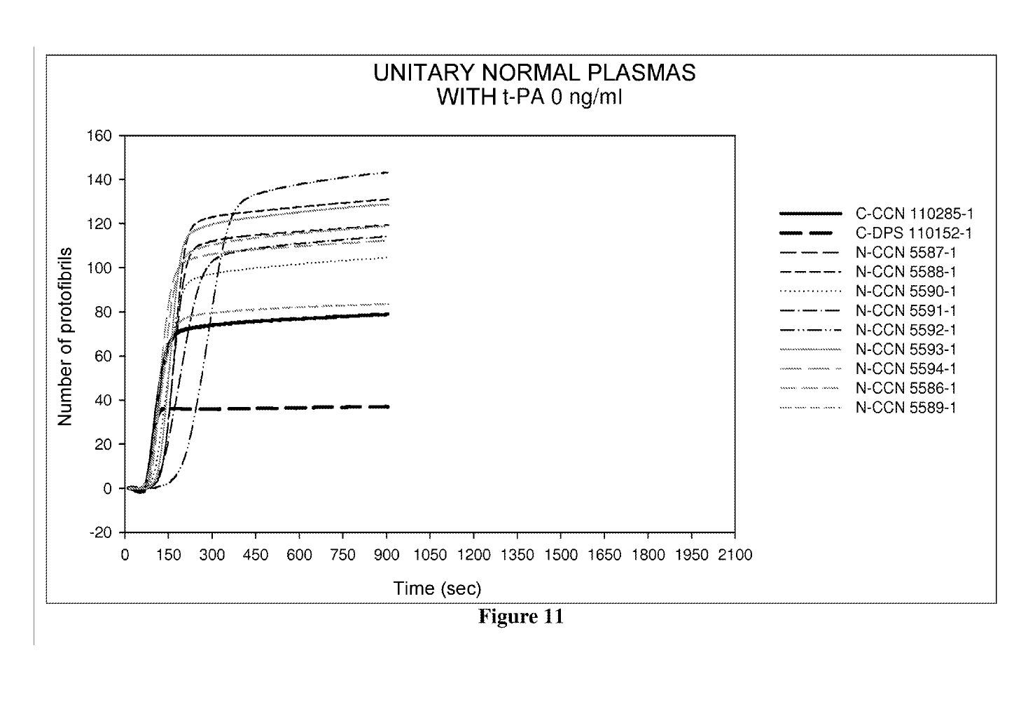

FIG. 11: Measurement of the number of protofibrils as a function of time for 9 normal plasmas (plasmas from N-CCN 5587 to N-CCN 5594), a normal control plasma (C-CCN) and a hypercoagulant control plasma (C-DPS), without t-PA.

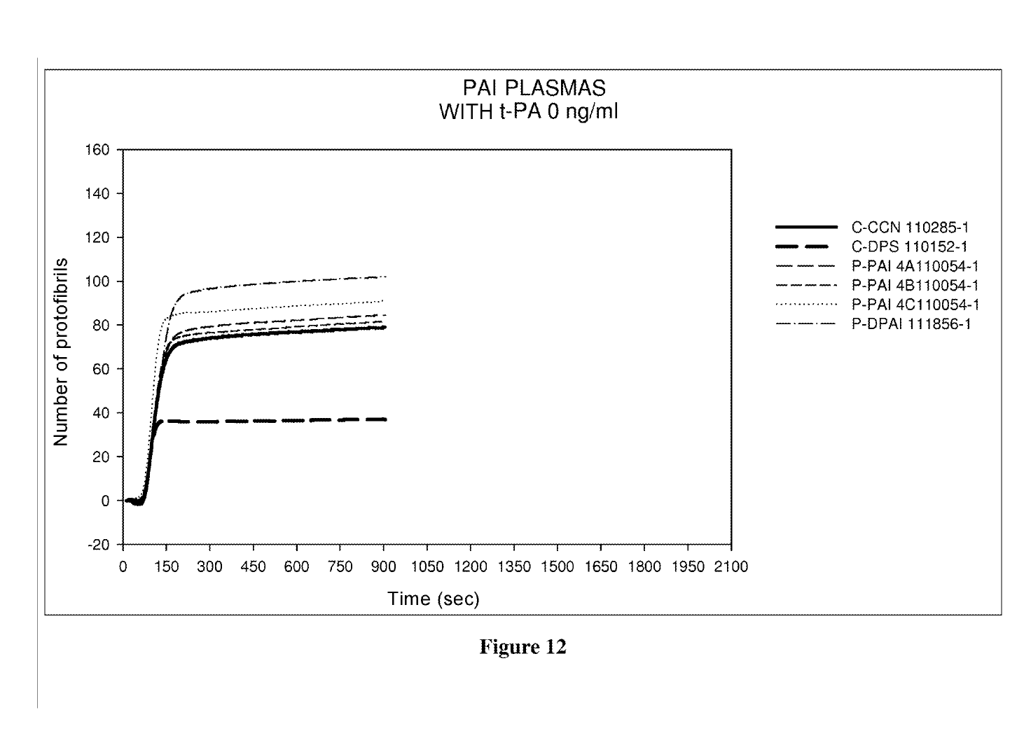

FIG. 12: Measurement of the number of protofibrils as a function of time for plasmas at 0, 20 and 40 AU/ml of PAI-1 (P-PAI 4A, 4B and 4C respectively), a plasma deficient in PAI-1 (P-DPAI), a normal control plasma (C-CCN) and a hypercoagulant control plasma (C-DPS), without t-PA.

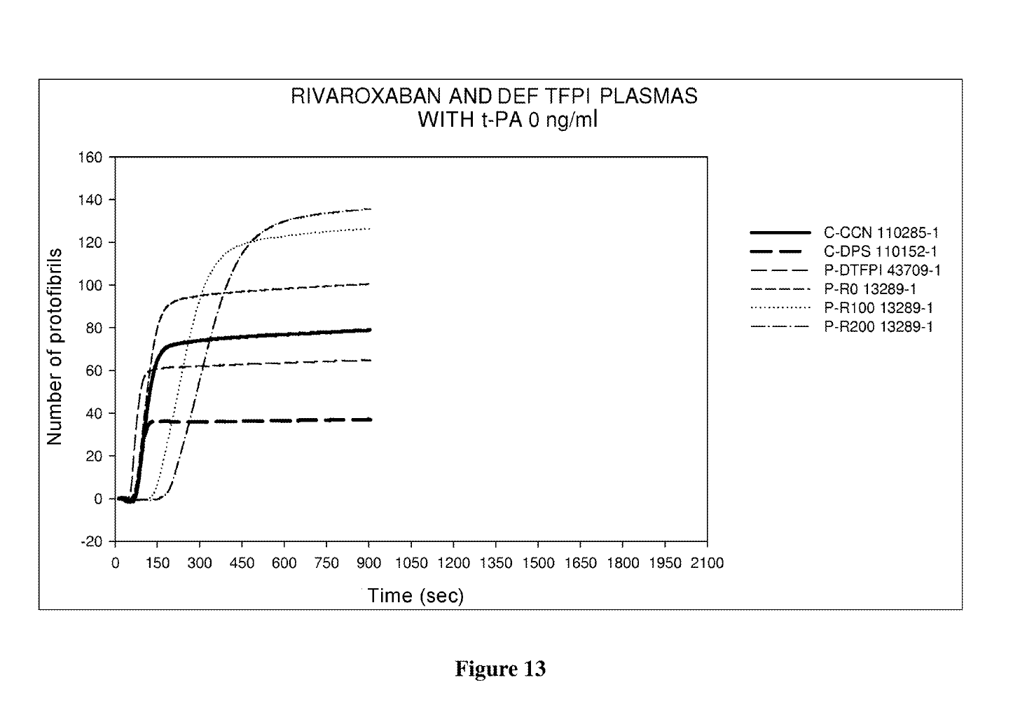

FIG. 13: Measurement of the number of protofibrils as a function of time for plasmas with an overload of rivaroxaban at 0, 100 and 200 ng/ml (P-R0, P-R100 and P-R200 respectively), a plasma deficient in TFPI (P-DTFPI), a normal control plasma (C-CCN) and a hypercoagulant control plasma (C-DPS), without t-PA.

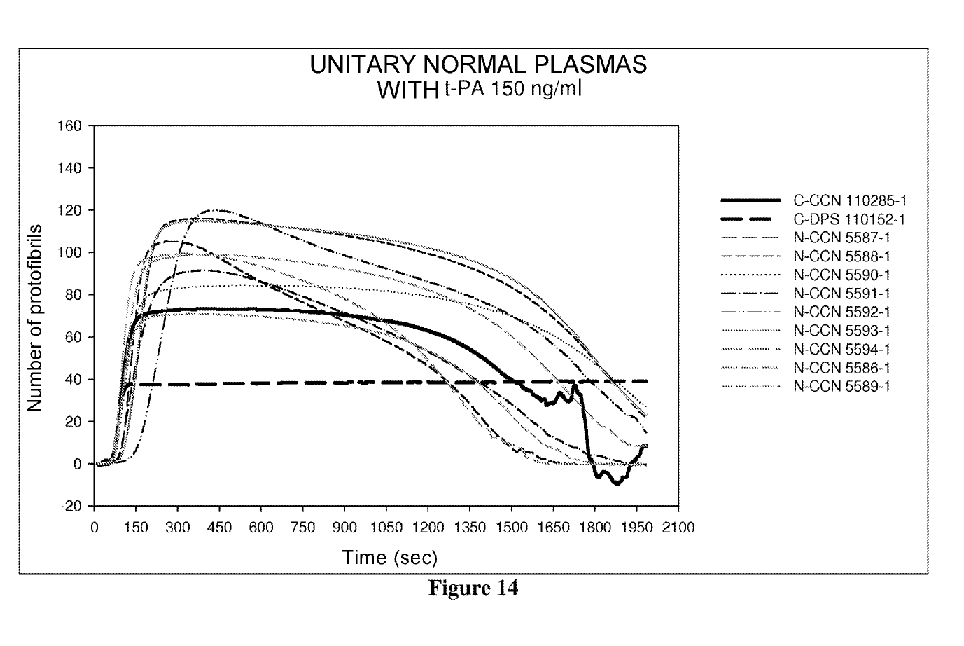

FIG. 14: Measurement of the number of protofibrils as a function of time for 9 normal plasmas (plasmas from N-CCN 5587 to N-CCN 5594), a normal control plasma (C-CCN) and a hypercoagulant control plasma (C-DPS), with t-PA at 150 ng/ml.

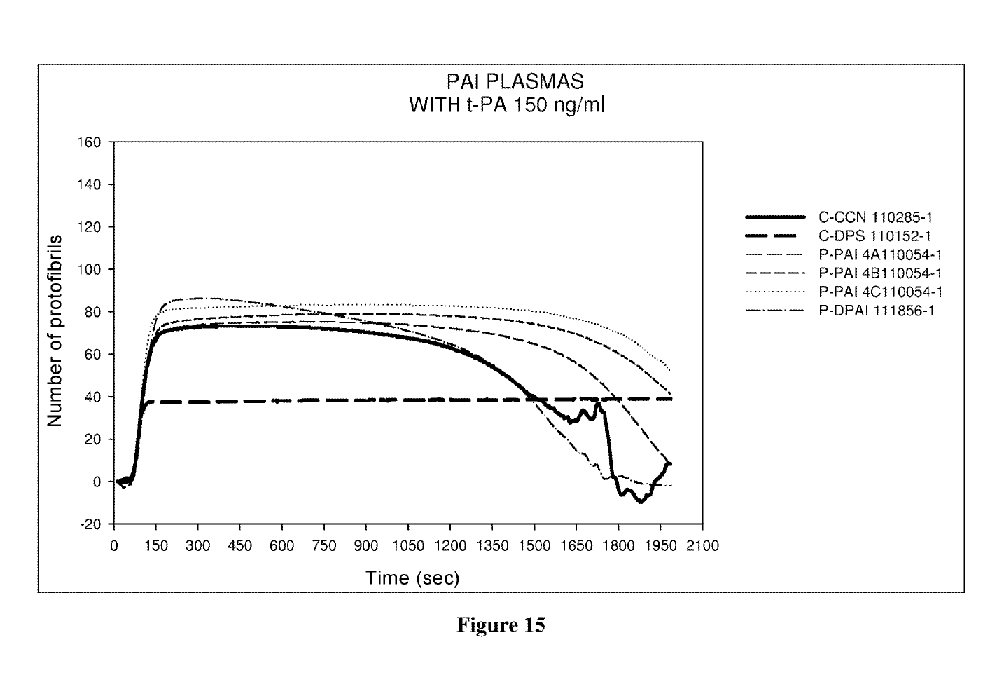

FIG. 15: Measurement of the number of protofibrils as a function of time for plasmas at 0, 20 and 40 AU/ml of PAI-1 (P-PAI 4A, 4B and 4C respectively), a plasma deficient in PAI-1 (P-DPAI), a normal control plasma (C-CCN) and a hypercoagulant control plasma (C-DPS), with t-PA at 150 ng/ml.

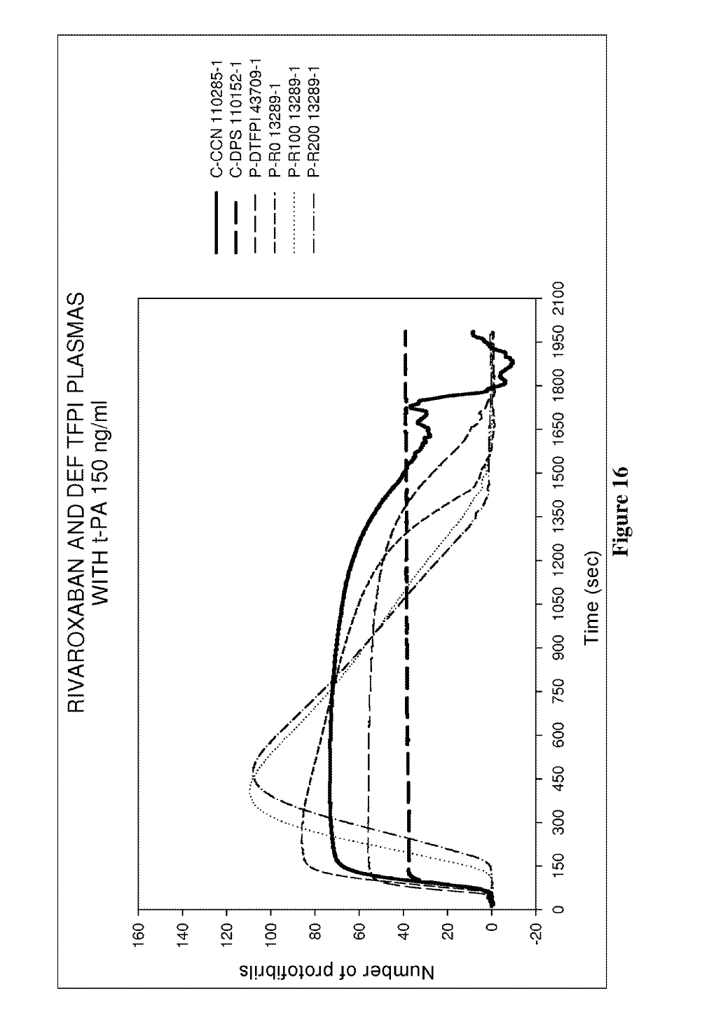

FIG. 16: Measurement of the number of protofibrils as a function of time for plasmas with an overload of rivaroxaban at 0, 100 and 200 ng/ml (P-R0, P-R100 and P-R200 respectively), a plasma deficient in TFPI (P-DTFPI), a normal control plasma (C-CCN) and a hypercoagulant control plasma (C-DPS), with t-PA at 150 ng/ml.

### EXAMPLES

### Implementation of the Method According to the Invention

### Experimental Protocols

The following protocols are used in the examples which follow:

Protocol A (Comparative, Manual Method):

The following are added manually to a 1 ml spectrophotometer cuvette:
667 μl of a sample of pure plasma,
167 μl of a [TF 1 to 5 pM final concentration+PL 4 μM final concentration] mixture.

This is then agitated manually.

It is incubated for 600 seconds at 37° C.

167 μl of CaCl₂ at a final concentration of 16.7 mM are added manually.

This is agitated manually.

Finally, the number of protofibrils is measured at 20 wavelengths of between 450 nm and 850 nm as a function of time for 90 minutes.

Protocol B (Without t-PA, Method on STA-R® with Tissue Factor):

To 8 cuvettes of the STA-R® Evolution Expert Series automated device (Stago), are added, simultaneously by the instrument:
200 μl of a sample of pure plasma,
50 μl of a [TF 2 to 5 pM final concentration+PL 4 μM final concentration] mixture.

The automated device agitates by means of the arm, and carries out an incubation for 300 seconds at 37° C.

It then adds 50 μl of CaCl₂ at a final concentration of 16.7 mM, and agitates by means of the needle of the initiating reagent.

Finally, the automated device measures the number of protofibrils at the 2 wavelengths of 540 nm and 780 nm as a function of time for 15 minutes.

Protocol C (With t-PA, Method on STA-R® with Tissue Factor and Plasminogen Activator):

To 8 cuvettes of the STA-R® Evolution Expert Series automated device (Stago), are added, simultaneously by the instrument:
200 μl of a sample of pure plasma,
50 μl of a [TF 2 to 5 pM final concentration+PL 4 μM final concentration+tPA 0.1 to 0.2 μg/ml final concentration] mixture.

The automated device agitates by means of the arm, and carries out an incubation for 300 seconds at 37° C.

It then adds 50 μl of CaCl₂ at a final concentration of 16.7 mM, and agitates by means of the needle of the initiating reagent.

Finally, the automated device measures the number of protofibrils at the 2 wavelengths of 540 nm and 780 nm as a function of time for 30 minutes.

### Example 1

### Structural Profile of Fibrin with 3 Normal, Hypocoagulant and Hypercoagulant Control Plasmas:

### 1a) Protocol A: Manual Method

The following are added manually to a 1 ml spectrophotometer cuvette:
667 μl of a sample of pure plasma: normal (frozen normal pool), hypocoagulant (heparinized control plasma 0.2 IU/ml) or hypercoagulant (protein S-depleted control plasma),
167 μl of a [TF 2 pM final concentration+PL 4 μM final concentration] mixture, followed by manual agitation.

It is incubated for 600 seconds at 37° C.

167 μl of CaCl₂ at a final concentration of 16.7 mM are added manually and the mixture is agitated manually.

Finally, the number of protofibrils is measured at 20 wavelengths of between 450 nm and 850 nm, as a function of time for 90 minutes.

The results are presented in FIG. 1.

The number of protofibrils measured as a function of time for the various normal, hypocoagulant and hypercoagulant plasmas makes it possible to distinguish these plasmas during thrombin generation and clot formation.

1b) Protocol B: Method on STA-R® with Tissue Factor TF

To 8 cuvettes of the STA-R® Evolution Expert Series automated device (Stago), are added, simultaneously by the instrument:
200 μl of a sample of pure: normal (Coag Control N control plasma and frozen normal pool), hypocoagulant (heparinized control plasma 0.2 IU/ml) and hypercoagulant (protein S-depleted plasma and plasma congenitally deficient in FVIII:C),
50 μl of a [TF 2 pM final concentration+PL 4 μM final concentration] mixture.

After agitation, and incubation for 300 seconds at 37° C., the automated device adds 50 μl of CaCl₂ at a final concentration of 16.7 mM, and agitates by means of the needle. Finally, the automated device measures the number of protofibrils at the 2 wavelengths of 540 nm and 780 nm as a function of time for 15 minutes.

The results are presented in FIG. 2.

The number of protofibrils of the various normal, hypocoagulant and hypercoagulant plasmas, as a function of time, makes it possible to distinguish these plasmas in less than 6 minutes in this example, during thrombin generation and clot formation.

The distinction is based on the number of protofibrils, the time to reach the plateau and the rate at which the plateau is reached.

1c) Protocol C: Method on STA-R® with Tissue Factor and Plasminogen Activator TF+t-PA—Influence of t-PA Concentration

To 8 cuvettes of the STA-R® Evolution Expert Series automated device (Stago), are added, simultaneously by the instrument:
200 μl of a sample of pure plasma: normal (Coag Control N control plasma and frozen normal pool), hypocoagulant (heparinized control plasma 0.2 IU/ml) and hypercoagulant (protein S-depleted plasma and plasma congenitally deficient in FVIII: C),
50 μl of a [TF 2 pM final concentration+PL 4 μM final concentration+t-PA 100, 150 and 175 ng/ml final concentration] mixture.

After agitation and incubation for 300 seconds at 37° C., the instrument adds 50 μl of CaCl₂ at a final concentration of 16.7 mM, and agitates by means of the needle.

Finally, the automated device measures the number of protofibrils at the 2 wavelengths of 540 nm and 780 nm as a function of time for 30 minutes.
a) [t-PA]=100 ng/ml

The results are presented in FIG. 3A.
b) [t-PA]=150 ng/ml

The results are presented in FIG. 3B.
c) [t-PA]=175 ng/ml

The results are presented in FIG. 3C.

The number of protofibrils of the various normal, hypocoagulant and hypercoagulant plasmas makes it possible to distinguish the plasmas in a time which depends on the t-PA concentration contained in the tissue factor. The distinction is based on the number of protofibrils, the time to reach the plateau and the rate at which the plateau is reached, and also the duration of the plateau and its slope.

The duration of the protofibril plateau, reflecting the stability of the clot, is reached in 30 minutes for the various profiles only starting from the concentration of 175 ng/ml of t-PA in this example.

The descending slope of the protofibril plateau is greater the faster the lysis; it is zero or small when the clot is resistant to lysis (deficient in factor FVIII:C, and protein S-depleted plasma respectively).

The disappearance of the protofibrils, corresponding to total lysis, is reached at 175 ng/ml oft-PA in this example.

### Example 2

### Structural Profile of Fibrin with the Normal Plasmas from Healthy Donors or from Patients with Normal Hemostasis Results

### 2a) Protocol A: Manual Method

The following are added manually to a 1 ml spectrophotometer cuvette:
667 μl of pure plasma sample of 4 frozen normal plasmas from healthy donors,
167 μl of a [TF 2 pM final concentration+PL 4 μM final concentration+t-PA 150 ng/ml] mixture, followed by manual agitation. It is incubated for 600 seconds at 37° C.

167 μl of CaCl₂ at a final concentration of 16.7 mM are added manually and agitation is carried out manually.

Finally, the number of protofibrils is measured at 20 wavelengths as a function of time for 90 minutes.

The results are presented in FIG. 4.

The number of protofibrils of the various normal plasmas, the time to reach the plateau and the rate at which the plateau is reached are very close. On the other hand, the duration of the plateau and its slope vary a great deal from one plasma to the other at the t-PA concentration of 150 ng/ml. The disappearance of the protofibrils is not achieved in 50 min for all the plasmas.

2b) Protocol C: Method on STA-R® with Tissue Factor and Plasminogen Activator TF+t-PA

To 8 cuvettes of the STA-R® Evolution Expert Series automated device (Stago), are added, simultaneously by the instrument:
200 μl of a sample of fresh pure plasma from patients deemed to be normal by means of routine hemostatis tests (quick time, partial thromboplastin time, fibrinogen),
50 μl of a [TF 2 pM final concentration+PL 4 μM final concentration+t-PA 150 ng/ml final concentration] optimized mixture.

After agitation and incubation for 300 seconds at 37° C., the instrument adds 50 μl of CaCl₂ at a final concentration of 16.7 mM, and agitates by means of the needle.

Finally, the automated device measures the number of protofibrils at the 2 wavelengths of 540 nm and 780 nm as a function of time for 30 minutes.

Hemostatis Results for the Fresh Plasmas from Patients “Deemed to be Normal by Means of Routine Hemostatis Tests”

| Plasmas from | Prothrombin | Partial thromboplastin | |
|---|---|---|---|
| normal patients | level (%) | time (sec) | Fibrinogen (g/l) |
| N-CCN 5586 | 100 | 40.5 | 3.80 |
| N-CCN 5587 | 99 | 34.4 | 2.30 |
| N-CCN 5588 | 100 | 31.1 | 4.00 |
| N-CCN 5589 | 96 | 35.7 | 3.60 |
| N-CCN 5590 | 94 | 32.7 | 3.30 |
| N-CCN 5591 | 100 | 37.9 | 2.90 |
| N-CCN 5592 | 100 | 37.8 | 3.00 |
| N-CCN 5593 | 99 | 31.4 | 3.70 |
| N-CCN 5594 | 92 | 32.6 | 3.50 |

The results are also presented in FIG. 5.

Number of Protofibrils

The number of protofibrils of the various fresh normal plasmas varies from 70 to 120;

the time to reach the plateau and the rate at which the plateau is reached vary more than for the healthy donors, in connection with the hemorrhagic or thrombotic risk associated with their hospitalization.

The duration of the plateau and its slope vary less than for the healthy donors, in connection with the optimized t-PA concentration in this example.

The disappearance of the protofibrils is reached in 33 min at the t-PA concentration used for all the plasmas. All these plasmas are distinguished from the protein S-depleted hypercoagulant plasma on the basis of the protofibril plateau.

### Example 3

### Structural Profile of Fibrin with 3 Normal, Hypocoagulant and Hypercoagulant Control Plasmas: Influence of the Wavelength

Protocol A was carried out as follows:

The following are added manually to a 1 ml spectrophotometer cuvette:
667 μl of pure plasma sample of the normal plasma (frozen normal pool), hypocoagulant plasma (heparinized control plasma 0.2 IU/ml) and hypercoagulant plasma (protein S-depleted plasma),
167 μl of a [TF 2 pM final concentration+PL 4 μM final concentration] mixture, followed by manual agitation.

It is incubated for 600 seconds at 37° C.

167 μl of CaCl₂ are added manually to a final concentration of 16.7 mM and manual agitation is carried out.

Finally, the number of protofibrils is measured at 2, 3, 7 or 20 wavelengths as a function of time for 90 minutes.

The results obtained at various numbers of wavelengths are then compared.

The results are presented in FIG. 6.

The curves are superimposable for each of the plasmas regardless of the number of wavelengths. The number of a minimum of 2 wavelengths was thus selected for the automated method on the routine instrument.

### Example 4

### Structural Profile of Fibrin with 3 Normal, Hypocoagulant and Hypercoagulant Control Plasmas: Repeatability and Reproducibility of the Automated Method on STA-R®

Protocol B on STA-R® with tissue factor TF was used as follows:

To 8 cuvettes of the STA-R® Evolution Expert Series automated device (Stago), are added, simultaneously by the instrument:
200 μl of normal (frozen normal plasma), hypocoagulant (heparinized control plasma 0.2 IU/ml, STA HEP C2) and hypercoagulant (protein S-depleted plasma, STA DEF PS) pure plasma sample,
50 μl of a [TF 2 pM final concentration+PL 4 μM final concentration] mixture.

After agitation, and incubation for 300 seconds at 37° C., the automated device adds 50 μl of CaCl₂ at a final concentration of 16.7 mM, and agitates by means of the needle. Finally, the automated device measures the number of protofibrils at the 2 wavelengths of 540 nm and 780 nm as a function of time for 15 minutes.

### 4a) Repeatability of the Method

24 tests were carried out in 3 consecutive assay series:

The results are in the table hereinafter.

The coefficients of variation (CV) obtained on the structural parameters of fibrin: number of protofibrils, radius and slope, are less than 2.5% for the normal plasma and less than or equal to 5% for the pathological plasmas, and therefore much lower than those obtained by means of the thrombin generation test. The CV on the time parameters (coagulation time, gel time and arrest time) are higher, in particular the arrest time, but they are not used for determining the structure of the clot, or for the interpretation, in contrast to the turbidimetric methods used to measure fibrin formation or lysis time.

### 4b) Reproducibility of the Method

20 measurements were carried out in 10 series of 2 measurements, for 5 consecutive days.

| Reproducibility of | | | | | | | | |
|---|---|---|---|---|---|---|---|---|
| the method: | | | | | | | NUMBER OF | |
| n = 10 series of 2 | | DOD | DOD | | | | PROTOFIBRILS | |
| measurements | | 540 nm | 780 nm | TCOAG | TGEL | TARREST | TARREST | TMAX |
| Normal | Mean | 0.617 | 0.301 | 80.6 | 103 | 372 | 77.0 | 81.9 |
| | Standard | 0.009 | 0.006 | 2.439 | 4.646 | 64.806 | 2.212 | 2.359 |
| | deviation | | | | | | | |
| | CV | 1.5% | 2.1% | 3.0% | 4.5% | 17.4% | 2.9% | 2.9% |
| Hypocoagulant | Mean | 0.684 | 0.394 | 150.3 | 219 | 745 | 103.4 | 104.6 |
| | Standard | 0.010 | 0.011 | 4.961 | 11.803 | 57.846 | 3.158 | 3.133 |
| | deviation | | | | | | | |
| | CV | 1.5% | 2.7% | 3.3% | 5.4% | 7.8% | 3.1% | 3.0% |
| Hypercoagulant | Mean | 0.398 | 0.181 | 76.9 | 97 | 157 | 48.0 | 49.7 |
| | Standard | 0.005 | 0.004 | 1.719 | 3.358 | 5.749 | 1.208 | 1.461 |
| | deviation | | | | | | | |
| | CV | 1.3% | 2.0% | 2.2% | 3.5% | 3.7% | 2.5% | 2.9% |

| Reproducibility of | | | | | | |
|---|---|---|---|---|---|---|
| the method: | | | | | | SLOPE |
| n = 10 series of 2 | | MEAN RADIUS | | DENSITY | | AROUND |
| measurements | | TARREST | TMAX | TARREST | TMAX | TGEL |
| Normal | Mean | 83.3 | 83.5 | 0.051 | 0.054 | 1.052 |
| | Standard | 0.702 | 0.899 | 0.001 | 0.001 | 0.044 |
| | deviation | | | | | |
| | CV | 0.8% | 1.1% | 2.2% | 1.9% | 4.2% |
| Hypocoagulant | Mean | 91.7 | 91.8 | 0.056 | 0.057 | 0.492 |
| | Standard | 0.608 | 0.606 | 0.001 | 0.001 | 0.014 |
| | deviation | | | | | |
| | CV | 0.7% | 0.7% | 2.1% | 1.9% | 2.8% |
| Hypercoagulant | Mean | 79.3 | 79.1 | 0.035 | 0.036 | 1.077 |
| | Standard | 0.713 | 1.034 | 0.001 | 0.000 | 0.040 |
| | deviation | | | | | |
| | CV | 0.9% | 1.3% | 1.5% | 1.4% | 3.7% |

Np: Number of protofibrils
Gel time: curve inflection point
Coagulation time: extrapolation on the X-axis or tangent to the inflection point
Arrest time: time at which the reaction rate corresponds to 1% of the maximum rate

The coefficients of variation (CV) obtained on the structural parameters of fibrin: number of protofibrils, radius and slope, are all less than 5% regardless of the plasma. The CVs on the time parameters (coagulation time, gel time and arrest time) are higher, in particular the arrest time, but they are not used for determining the structure of the clot, or for the interpretation, in contrast to the turbidimetric methods used to measure fibrin formation or lysis time.

### Example 5

### Structural Profile of Fibrin with 4 Normal, Hypocoagulant and Hypercoagulant Plasmas: Influence of Variations in Fibrinogen of the Patient on the Determination of the Profile Thereof

Protocol B on STA-R® with tissue factor TF was used as follows:

To 8 cuvettes of the STA-R® Evolution Expert Series automated device (Stago), are added, simultaneously by the instrument:
200 μl of normal (frozen normal plasma and frozen normal pool), hypocoagulant (heparinized control plasma 0.2 IU/ml) and hypercoagulant (protein-S depleted plasmas) pure plasma sample,
50 μl of a [TF 2 pM final concentration+PL 4 μM final concentration] mixture.

After agitation, and incubation for 300 seconds at 37° C., the automated device adds 50 μl of CaCl₂ at a final concentration of 16.7 mM, and agitates by means of the needle. Finally, the automated device measures the number of protofibrils at the 2 wavelengths of 540 nm and 780 nm as a function of time for 15 minutes.

The calculations are carried out for each plasma with variations in fibrinogen level of plus or minus 20% in order to study the influence of these variations on the determination of the profile of each plasma.

The results are presented in FIGS. 7 to 10.

The optical density and also the structure of the fibrin depend on the intrinsic fibrinogen level of the sample. The optical density and also the number of protofibrils and the density increase all the more as the fibrinogen increases according to a linear relationship y=ax−b.

### Example 6

### Structural Profile of Fibrin with Normal, Hypocoagulant, Hypercoagulant, Hypofibrinolytic and Hyperfibrinolytic Plasmas: Distinction Between Normal Samples and Pathological Samples

6a) Protocol B on STAR® with Tissue Factor TF:

To 8 cuvettes of the STA-R® Evolution Expert Series automated device (Stago), are added, simultaneously by the instrument:
200 μl of a sample of pure plasma from 9 patients with normal hemostatis results, of a frozen plasma deficient in PAI-1, of 3 control plasmas at 0, 20 and 40 AU/ml of PAI-1, of a protein S-deficient plasma, of a TFPI-depleted plasma and of 3 plasmas with an overload of rivaroxaban at 0, 100 and 200 ng/ml,
50 μl of a [TF 2 to 5 pM final concentration+PL 4 μM final concentration] mixture.

The automated device agitates by means of the arm, and carries out an incubation for 300 seconds at 37° C.

It then adds 50 μl of CaCl₂ at a final concentration of 16.7 mM, and agitates by means of the needle.

Finally, the automated device measures the number of protofibrils at the 2 wavelengths of 540 nm and 780 nm as a function of time for 15 minutes.

The results are presented in FIGS. 11 to 13.

The number of protofibrils of the various normal, hypocoagulant and hypercoagulant plasmas makes it possible to distinguish these plasmas in 15 minutes; on the other hand, it does not make it possible to distinguish the hypofibrinolytic and hyperfibrinolytic plasmas from the other plasmas.

The distinction of the hypercoagulant, normal and hypocoagulant profiles is based on the number of protofibrils, the time to reach the plateau and the rate at which the plateau is reached:
these parameters increase in parallel to the concentration of anticoagulant, attesting to a looser structure of the clot;
they are lower for the hypercoagulant plasmas than for all the normal plasmas, attesting to a more compact structure of the clot;
these parameters are included in the range of the normal samples for the hypofibrinolytic plasmas with an overload of PAI-1. The information obtained during the thrombin generation is insufficient and does not make it possible to distinguish this type of plasma.
   6b) Protocol C on STA-R® with Tissue Factor and Plasminogen Activator TF+t-PA:

To 8 cuvettes of the STA-R® Evolution Expert Series automated device (Stago), are added, simultaneously by the instrument:
200 μl of sample of pure plasma from 9 patients with normal hemostatis results, of a frozen plasma deficient in PAI-1, of 3 control plasmas at 0, 20 and 40 AU/ml of PAI-1, of a protein S-depleted plasma, of a TFPI-depleted plasma and of 3 plasmas with an overload of rivaroxaban at 0, 100 and 200 ng/ml,
50 μl of a [TF 2 to 5 pM final concentration+PL 4 μM final concentration+t-PA 150 ng/ml] mixture.

The automated device agitates by means of the arm, and carries out an incubation for 300 seconds at 37° C.

It then adds 50 μl of CaCl₂ at a final concentration of 16.7 mM, and agitates by means of the needle.

Finally, the automated device measures the number of protofibrils at the 2 wavelengths of 540 nm and 780 nm as a function of time for 30 minutes.

The results are presented in FIGS. 14 to 16.

The number of protofibrils of the various normal, hypocoagulant, hypercoagulant, hypofibrinolytic and hyperfibrinolytic plasmas makes it possible to distinguish all these plasmas in 30 minutes on the basis of their structural profile during thrombin generation, fibrin formation and lysis.

The distinction of all the profiles is obtained on the basis of the number of protofibrils, the time to reach the plateau and the rate at which the plateau is reached, the duration of the plateau and its slope.

The protofibril plateau, reflecting the stability of the clot, lasts for more than 20 min, 10 to 20 min and less than 10 min for a hypercoagulant profile, a normal profile and a hypocoagulant profile, respectively:
the protofibril plateau is shortened proportionally to the concentration of anti-FXa anticoagulant (rivaroxaban) of the hypocoagulant plasma, in connection with the hemorrhagic risk due to the anticoagulant;
it is extended proportionally to the concentration of plasminogen activator inhibitor (PAI-1) of the hypofibrinolytic plasma, and with the deficiencies in protein S and TFPI, in connection with the thrombotic risk associated with these hemostasis disorders.

The downward slope of the protofibril plateau is all the greater the faster the lysis, this is the case with the hyperfibrinolytic plasmas (deficient in PAI-1 or containing rivaroxaban); it is zero when the clot is resistant to lysis, which is the case with the hypercoagulant plasmas (deficient in protein S or in tissue factor inhibitor TFPI).

The disappearance of the protofibrils, corresponding to total lysis, is reached in 33 min for all the samples, except for one of the hypercoagulant plasmas, deficient in protein S.

1-17. (canceled)

18. A method for determining the structural profile of a fibrin clot, reflecting the stability thereof in a biological sample from a patient, said method comprising steps of:
a) mixing the undiluted biological sample with tissue factor and phospholipids;
b) incubating the mixture obtained in step a), then adding calcium ions to the mixture obtained, in order to initiate the formation of a clot;
c) measuring the turbidity or the optical density of the clot being formed in step b), at least two wavelengths of between 450 nm and 850 nm, and for a time of between 1 and 35 minutes; and
d) determining the structural profile of the clot analyzed in c) expressed as number of protofibrils, density and radius, and calculated by means of the formula
τ.λ⁵=A[Fg].(λ²−B),
wherein τ is the turbidity of the clot or the expression of the optical density in turbidity, at a given wavelength λ, [Fg] is the initial weight concentration of fibrinogen, and A and B are coefficients proportional to the density and to the radius of the fibers constituting the clot, respectively.

19. The method according to claim 18, wherein in step a), the tissue factor and the phospholipids are premixed with a solution of plasminogen activator, then the whole mixture is added to the undiluted biological sample.

20. The method according to claim 19, wherein the plasminogen activator is tissue plasminogen activator.

21. The method according to claim 19, wherein the tissue factor of the mixture of step a) is present in an amount such that its final concentration in the mixture with the undiluted biological sample is between 0.01 and 20 pM, or between 0.1 and 5 pM, or between 1 and 5 pM.

22. The method according to claim 19, wherein mixing the tissue factor with the tissue plasminogen activator is carried out in a [t-PA/TF] respective weight ratio of between 800 and 1700, or in a ratio of 75-150 ng/ml of t-PA for 0.1-5 pM of TF.

23. The method according to claim 22, wherein mixing the tissue factor with the tissue plasminogen activator is carried out in a ratio of 75-150 ng/ml of t-PA for 1-5 pM of TF.

24. The method according to claim 18, wherein the mixture of step a) comprises at least one divalent cation.

25. The method according to claim 24, wherein the at least one divalent cation is calcium ions.

26. The method according to claim 18, wherein the biological sample is a blood sample, a plasma sample, a sample of platelet-rich plasma or of platelet-poor plasma, or a sample of plasma containing platelet microparticles, erythrocytes or any other cell.

27. The method according to claim 26, wherein the biological sample is a sample of platelet-poor plasma.

28. The method according to claim 18, wherein the biological sample has a volume of between 5 μl and 500 μl, or of between 50 μl and 400 μl, or of between 50 μl and 300 μl, or of between 100 μl and 300 μl, or of approximately 200 μl.

29. The method according to claim 19, wherein the plasminogen activator has an affinity for fibrin.

30. The method according to claim 18, wherein step b) comprises incubating the mixture obtained in step a) for a time of between 60 seconds and 400 seconds, or of between 200 seconds and 350 seconds, at a temperature of between 30° C. and 40° C.

31. The method according to claim 18, wherein step b) initiates the thrombin generation and the formation of a clot.

32. The method according to claim 18, wherein measuring the turbidity or the optical density of step c) is carried out at at least two wavelengths closest to the extremes.

33. The method according to claim 32, wherein the two wavelengths closest to the extremes are 540 nm and 760 nm.

34. The method according to claim 32, wherein measuring the turbidity or the optical density of step c) is carried out at at least two wavelengths closest to the extremes simultaneously

35. The method according to claim 18, wherein measuring the turbidity or the optical density of step c) is carried out for a time of between 1 minute and 35 minutes, or of approximately 15 minutes with tissue factor alone, or of approximately 30 minutes with a mixture of tissue factor and tissue plasminogen activator.

36. The method according to claim 18, wherein at least steps c) and d) are carried out on an automated diagnostic device.

37. The method according to claim 36, wherein the automated diagnostic device is a coagulation analyzer.

38. A method for predicting the risk of bleeding, thrombosis or rethrombosis using a biological sample from a patient, said method comprising following steps of:
a) mixing the undiluted biological sample with tissue factor and phospholipids;
b) incubating the mixture obtained in step a), then adding calcium ions to the mixture obtained, in order to initiate the formation of a clot;
c) measuring the turbidity or the optical density of the clot being formed in step b), at at least two wavelengths of between 450 nm and 850 nm, and for a time of between 1 and 35 minutes; and
d) determining the profile of the clot analyzed in c) by means of the formula
τ.λ⁵=A[Fg].(λ²−B),
wherein τ is the turbidity of the clot at a given wavelength A, [Fg] is the initial weight concentration of fibrinogen, and A and B are coefficients proportional to the density and to the radius of the fibers constituting the clot, respectively, and
e) comparing the profile obtained in d) with a control.

39. The method according to claim 38, further comprising a step f), after step e), of selecting the anticoagulant most suitable for the clinical situation of said patient, said clinical situation being chosen from atrial fibrillation or another cardiac impairment, a cancer or another malignant condition or precancerous state, and a risk of venous or arterial thrombosis.

40. The method according to claim 38, wherein the control of step e) is chosen from:
a biological sample from one or more healthy individuals, preferably a reference plasma,
a biological sample from one or more quality controls for mimicking the conditions of patients,
a biological sample from a hemorrhagic patient,
a biological sample from a thrombotic patient, and
a biological sample from a patient having suffered rethrombosis.
